SEQUENCE LISTING (11 sequences)

SEQ ID NO: 1 - DNA, 707 bp, Mus musculus
  source 1..707: mol_type = genomic DNA
gtaagagttttatgttttttcatctctgcttgtatttttctagtaatggaagcctggtattttaaaatagttaaattttcctttagtgctgatttctagattattattactgttgttgttgttattattgtcattatttgcatctgagaacccttaggtggttatattattgatatatttttggtatctttgatgacaataatgggggattttgaaagcttagctttaaatttcttttaattaaaaaaaaatgctaggcagaatgactcaaattacgttggatacagttgaatttattacggtctcatagggcctgcctgctcgaccatgctatactaaaaattaaaagtgtgtgttactaattttataaatggagtttccatttatatttacctttatttcttatttaccattgtcttagtagatatttacaaacatgacagaaacactaaatcttgagtttgaatgcacagatataaacacttaacgggttttaaaaataataatgttggtgaaaaaatataactttgagtgtagcagagaggaaccattgccaccttcagattttcctgtaacgatcgggaactggcatcttcagggagtagcttaggtcagtgaagagaagaacaaaaagcagcatattacagttagttgtcttcatcaatctttaaatatgttgtgtggtttttctctccctgtttccacag

SEQ ID NO: 2 - DNA, 21 bp, Mus musculus
  source 1..21: mol_type = genomic DNA
gatcgggaactggcatcttca

SEQ ID NO: 3 - DNA, 27 bp, Mus musculus
  source 1..27: mol_type = genomic DNA
gatcgggaactggcatcttcagggagt

SEQ ID NO: 4 - DNA, 21 bp, Mus musculus
  source 1..21: mol_type = genomic DNA
acatgacagaaacactaaatc

SEQ ID NO: 5 - DNA, 27 bp, Mus musculus
  source 1..27: mol_type = genomic DNA
acatgacagaaacactaaatcttgagt

SEQ ID NO: 6 - DNA, 23 bp, Homo sapiens
  source 1..23: mol_type = genomic DNA
taaagcatagtgcaatggatagg

SEQ ID NO: 7 - DNA, 23 bp, Homo sapiens
  source 1..23: mol_type = genomic DNA
gagcaacctcactcttgtctggg

SEQ ID NO: 8 - DNA, 21 bp, Mus musculus
  source 1..21: mol_type = genomic DNA
aagtggcattccatgtgtcct

SEQ ID NO: 9 - DNA, 21 bp, Mus musculus
  source 1..21: mol_type = genomic DNA
gtgagtgatctcaattgattt

SEQ ID NO: 10 - DNA, 21 bp, Mus musculus
  source 1..21: mol_type = genomic DNA
gagcaaccttctaggtttaag

SEQ ID NO: 11 - DNA, 21 bp, Mus musculus
  source 1..21: mol_type = genomic DNA
gctaagcatccttagcttggt

(19) United States
(12) Patent Application Publication
(10) Pub. No.: US 2026/0132389 A1
(43) Pub. Date: May 14, 2026

(54) MATERIALS AND METHODS FOR REDOSING SUBJECTS RECEIVING GENE INSERTION THERAPEUTICS

(71) Applicant: BLUEALLELE CORPORATION, Oakdale, MN (US)

(72) Inventor: Nicholas J. BALTES, Oakdale, MN (US)

(73) Assignee: BLUEALLELE CORPORATION, Oakdale, MN (US)

(21) Appl. No.: 18/947,288

(22) Filed: Nov. 14, 2024

(51) Int. Cl. C12N 9/22; C12N 15/10; C12N 15/11

(52) U.S. Cl. CPC C12N 9/22; C12N 15/102; C12N 15/111; C12N 2310/20

(57) ABSTRACT

The present document is in the field of genome editing. In some embodiments, this document relates to the targeted insertion of DNA using rare-cutting endonucleases.

### TECHNICAL FIELD

The present document is in the field of genome editing. More specifically, this document relates to the targeted insertion of DNA using rare-cutting endonucleases.

### REFERENCE TO A SEQUENCE LISTING XML

This application contains a Sequence Listing which has been submitted electronically in XML format. The Sequence Listing XML is incorporated herein by reference. Said XML file, created on Dec. 9, 2024, is named BA2024-16-TRACK ONE_SL.xml and is 11,208 bytes in size.

### BACKGROUND

Monogenic disorders are caused by one or more mutations in a single gene, examples of which include sickle cell disease (hemoglobin-beta gene), cystic fibrosis (cystic fibrosis transmembrane conductance regulator gene), and Tay-Sachs disease (beta-hexosaminidase A gene). Monogenic disorders have been an interest for gene editing therapies, as replacement of the defective sequence with a corrective nucleic acids could provide therapeutic benefits. However, one bottleneck for generating effective gene editing therapies includes low insertion efficiencies of corrective nucleic acids. Methods to increase the number of insertion events within a target population of cells may prove valuable for overcoming therapeutic thresholds.

### SUMMARY

Gene editing holds promise for correcting mutations found in genetic disorders; however, many challenges remain for creating effective therapies for individual disorders, including those that are caused by gain-of-function mutations, or where precise repair is required. These challenges include achieving insertion frequencies of corrective DNA at frequencies sufficient for overcoming the therapeutic threshold.

The methods described herein provide novel approaches for increasing the number of cells in a population with desired insertion events. The disclosure herein is based at least in part on the design of methods that enable the redosing of a population of cells to increase the number of cells with a desired insertion event. The method includes a two-step process, wherein the first step involves integrating an insertion template into the genome of a population of cells using a rare-cutting endonuclease targeting a first target site. After waiting a period of time to allow for the insertion template to integrate into the first target site, the second step involves re-administering the same insertion template with a second rare-cutting endonuclease targeting a second target site. The second target site can be adjacent to, either upstream or downstream of, the first target site. Integration of the insertion template at the second target site further increases the number of cells within the population with a desired gene insertion event. Integration of the template at either the first or second target site results in a functional gene edit that contributes to the overall efficacy of the therapeutic approach. The methods are particularly useful in cases where high levels of gene insertion are required. The methods described herein can be used for applied research (e.g., gene editing therapeutics) or basic research (e.g., editing of animal models, or understanding gene function).

The methods presented herein solve the challenge of low efficiency gene insertion in a population of cells when seeking to express a coding sequence of interest. Using the methods presented herein, the number of gene insertion events within a population of cells can be increased, and the same coding sequence of interest can be expressed after redosing. This method can be particularly useful in cases where subjects receive a first gene-insertion therapeutic, but the efficacy did not overcome a therapeutic threshold. This method can also be useful in cases where subjects receive a first gene-insertion therapeutic, but the protein expression decreased over time. The method can result in increases in the number of cells, within a population of cells, with a desired gene insertion event. By using the methods presented herein, the number of cells with a desired insertion event can increase by 25% or more, 50% or more, 100% or more, or 200% or more after redosing, and relative to the insertion event numbers after the first dose. By using the methods presented herein, the level of desired protein production can increase by 25% or more, 50% or more, 100% or more, or 200% or more after redosing, and relative to the expression levels of the first dose.

The methods described herein are compatible with current in vivo delivery vehicles (e.g., adeno-associated virus vectors and lipid nanoparticles), and they address several challenges with achieving high-frequency gene insertion.

In one embodiment, this document features a method of treating a genetic disease in a subject comprising a population of cells comprising a first and second target site in their genome, the method comprising administering to the subject a first composition comprising an insertion template and a first rare-cutting endonuclease that targets a first target site; waiting a period of time to allow for the insertion template to integrate into the first target site of a population of cells in the subject, wherein the period of time is between 1 day and 20 years; administering to the subject a second composition comprising the insertion template and a second rare-cutting endonuclease, whereby the insertion template is integrated into the genome of the population of cells and/or their progeny at the second target site. The second composition can be administered 1 day or more, 7 days or more, 1 month or more, or 1 year or more after the first composition. The methods are compatible with rare-cutting endonucleases, including TALENs, CRISPR, and zinc-finger nucleases. The CRISPR endonuclease can be a Cas9 endonuclease. The first target site can be within an intron of an endogenous gene within a cell of the subject. The second target site can be within an intron of an endogenous gene within a cell of the subject. Both the first and second target sites can be within the same intron of the endogenous gene. The second target site can be either upstream or downstream of the first target site. The second target site can be separated from the first target site by at least 1 nucleotide, at least 10 nucleotides, at least 100 nucleotides, or at least 1000 nucleotides. In certain embodiments, the first target site can be within the first intron of the albumin gene. In an embodiment, the first target site can comprise the sequence TAAAGCATAGTGCAATGGATAGG (SEQ ID NO:6). In other embodiments, the first target site can be within intron 4 of the RHO gene. In one embodiment, the same insertion template is integrated into both the first and second target sites. The insertion template can comprise a splice acceptor sequence operably linked to a coding sequence. In other embodiments, the insertion template can comprise a coding sequence operably linked to a splice donor sequence. In preferred embodiments, the same insertion template is used for both the first and second steps in the method; or alternatively, two different insertion templates can be used for the first and second steps, however, the insertion templates preferably and ultimately should perform the same function after insertion, which includes the same transcription regulation along with final expression of the same protein product. If there are differences in the insertion template, the differences can include differing flanking sequences used to integrate the template (e.g., homology arms), or differences in the nucleic acid sequences that encode the splice acceptors, coding sequences (i.e., through codon degeneracy) or splice donors.

In another embodiment, this document features a method of increasing the frequency of gene insertion events within a population of cells, the method comprising administering to the population cells a first composition comprising an insertion template and a first rare-cutting endonuclease that targets a first target site; waiting a period of time to allow for the insertion template to integrate into the first target site of the population of cells, wherein the period of time is between 1 day and 20 years; administering to the population of cells and/or their progeny a second composition comprising the insertion template and a second rare-cutting endonuclease, whereby the insertion template is integrated into the genome of the population of cells and/or their progeny at the second target site. The second composition can be administered 1 day or more, 7 days or more, 1 month or more, or 1 year or more after the first composition. The methods are compatible with rare-cutting endonucleases, including TALENs, CRISPR, and zinc-finger nucleases. The CRISPR endonuclease can be a Cas9 endonuclease. The first target site can be within an intron of an endogenous gene within a cell of the subject. The second target site can be within an intron of an endogenous gene within a cell of the subject. Both the first and second target sites can be within the same intron of the endogenous gene. The second target site can be either upstream or downstream of the first target site. The second target site can be separated from the first target site by at least 1 nucleotide, at least 10 nucleotides, at least 100 nucleotides, or at least 1000 nucleotides. In certain embodiments, the first target site can be within the first intron of the albumin gene. In an embodiment, the first target site can comprise the sequence TAAAGCATAGTGCAATGGATAGG (SEQ ID NO:6). In other embodiments, the first target site can be within intron 4 of the RHO gene. In one embodiment, the same insertion template is integrated into both the first and second target sites. The insertion template can comprise a splice acceptor sequence operably linked to a coding sequence. In other embodiments, the insertion template can comprise a coding sequence operably linked to a splice donor sequence. In preferred embodiments, the same insertion template is used for both the first and second steps in the method; or alternatively, two different insertion templates can be used for the first and second steps, however, the insertion templates preferably and ultimately should perform the same function after insertion, which includes the same transcription regulation along with final expression of the same protein product. If there are differences in the insertion template, the differences can include differing flanking sequences used to integrate the template (e.g., homology arms), or differences in the nucleic acid sequences that encode the splice acceptors, coding sequences (i.e., through codon degeneracy) or splice donors.

In another embodiment, this document features A method of modifying the expression of a gene in a population of cells comprising a first and second target sites, the method comprising administering to the population cells a first composition comprising an insertion template and a first rare-cutting endonuclease that targets a first target site within an intron of the gene, waiting a period of time to allow for the insertion template to integrate into the first target site of the population of cells, wherein the period of time is between 1 day and 20 years, administering to the population of cells and/or their progeny a second composition comprising the insertion template and a second rare-cutting endonuclease, whereby the insertion template is integrated within the intron of the gene at the second target site. The second composition can be administered 1 day or more, 7 days or more, 1 month or more, or 1 year or more after the first composition. The methods are compatible with rare-cutting endonucleases, including TALENs, CRISPR, and zinc-finger nucleases. The CRISPR endonuclease can be a Cas9 endonuclease. The first target site can be within an intron of an endogenous gene within a cell of the subject. The second target site can be within an intron of an endogenous gene within a cell of the subject. Both the first and second target sites can be within the same intron of the endogenous gene. The second target site can be either upstream or downstream of the first target site. The second target site can be separated from the first target site by at least 1 nucleotide, at least 10 nucleotides, at least 100 nucleotides, or at least 1000 nucleotides. In certain embodiments, the first target site can be within the first intron of the albumin gene. In an embodiment, the first target site can comprise the sequence TAAAGCATAGTGCAATGGATAGG (SEQ ID NO:6). In other embodiments, the first target site can be within intron 4 of the RHO gene. In one embodiment, the same insertion template is integrated into both the first and second target sites. The insertion template can comprise a splice acceptor sequence operably linked to a coding sequence. In other embodiments, the insertion template can comprise a coding sequence operably linked to a splice donor sequence. In preferred embodiments, the same insertion template is used for both the first and second steps in the method; or alternatively, two different insertion templates can be used for the first and second steps, however, the insertion templates preferably and ultimately should perform the same function after insertion, which includes the same transcription regulation along with final expression of the same protein product. If there are differences in the insertion template, the differences can include differing flanking sequences used to integrate the template (e.g., homology arms), or differences in the nucleic acid sequences that encode the splice acceptors, coding sequences (i.e., through codon degeneracy) or splice donors.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety for all purposes. In case of conflict, the present specification, including definitions, will control. In addition, the materials, methods, and examples are illustrative only and not intended to be limiting.

The details of one or more embodiments of the invention are set forth in the description below. Other features, objects, and advantages of the invention will be apparent from the description and from the claims.

### DESCRIPTION OF DRAWINGS

FIG. 1 is an illustration of insertion templates that can be used for targeted insertion into the genome of cells. SA; splice acceptor site; CDS, coding sequence; T, terminator; SA1, splice acceptor site 1, CDS1, coding sequence 1; T1, terminator 1; SA2, splice acceptor site 2, CDS2, coding sequence 2; T2, terminator 2; SD, splice donor; SD1, splice donor 1; SD2, splice donor 2; P, promoter; P1, promoter 1; P2, promoter 2.

FIG. 2 is an exemplary illustration showing integration of an insertion template within an intron of an endogenous gene at target site 1, followed by integration of the insertion template at target site 2.

FIG. 3 is an exemplary illustration showing integration of an insertion template within an intergenic region at target site 1, followed by integration of the insertion template at target site 2.

FIG. 4 is an illustration of the outcomes after administering the insertion template with the rare-cutting endonuclease for target site 1. X represents a mutation introduced by the rare-cutting endonuclease that destroys the target site. A fraction of the population of cells are predicted to comprise an insertion event.

FIG. 5 is an illustration of the outcomes that occur with one of the cell populations from step 1, after the subsequent administration of the insertion template with a rare-cutting endonuclease targeting the second target site.

FIG. 6 is an exemplary illustration showing integration of an insertion template comprising a [SA]-[CDS]-[T] sequence at both target site 1 and target site 2. The same protein product is predicted from single insertions and double insertions.

FIG. 7 is an exemplary illustration showing integration of a bidirectional insertion template comprising a [SA]-[CDS]-[T]-[T]-[CDS]-[SA] sequence at both target site 1 and target site 2. The same protein product is predicted from single insertions and double insertions.

FIG. 8 are illustrations of the bidirectional insertion template, unidirectional insertion template, and SaCas9 vector for insertion into target site 1 of the mouse albumin gene.

FIG. 9 is a graph showing F9 expression after integration of the insertion templates into target site 1.

FIG. 10 is a gel image of PCRs to detect the 5′ or 3′ junctions of the bidirectional template within target site 1.

FIG. 11 are illustrations of the bidirectional insertion template and SaCas9 vector for insertion into target site 2 of the mouse albumin gene.

FIG. 12 are illustrations of the bidirectional insertion template and SaCas9 vectors for insertion into target site 1 and 2 of the mouse RHO gene.

FIG. 13 are images of retinal cells expressing report proteins after integration of insertion templates within target site 1 and target site 2 of the RHO gene.

FIG. 14 are illustrations of the bidirectional insertion template and SaCas9 vectors for insertion into target site 1 and 2 of the mouse ACTB gene.

FIG. 15 are images of central nervous system cells expressing report proteins after integration of insertion templates within target site 1 and target site 2 of the ACTB gene.

### DETAILED DESCRIPTION

Disclosed herein are methods and compositions for increasing the frequency of cells, within a population of cells, with a desired gene insertion event. In some embodiments, the methods include integrating an insertion template into a first target site within an endogenous gene in a population of cells. The method then includes the subsequent integration of the insertion template into a second target site within the endogenous gene, wherein the redosing event increases the frequency of insertion events within the population of cells. The method can be used within introns of endogenous genes, or within intergenic, safe harbor sites, across the genome. A particular benefit of this method is with patients with genetic diseases that receive a first gene insertion therapeutic, but the efficacy or durability was not sufficient for alleviating symptoms of the disease.

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

As used herein, the terms “nucleic acid” and “polynucleotide,” can be used interchangeably. Nucleic acid and polynucleotide can refer to a deoxyribonucleotide or ribonucleotide polymer, in linear or circular conformation, and in either single- or double-stranded form. These terms are not to be construed as limiting with respect to the length of a polymer. The terms can encompass known analogues of natural nucleotides, as well as nucleotides that are modified in the base, sugar and/or phosphate moieties.

The terms “polypeptide,” “peptide” and “protein” can be used interchangeably to refer to amino acid residues covalently linked together. The term also applies to proteins in which one or more amino acids are chemical analogues or modified derivatives of corresponding naturally occurring amino acids.

The terms “operatively linked” or “operably linked” are used interchangeably and refer to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence but need not be directly adjacent to it. For example, an enhancer is a transcriptional regulatory sequence that is operatively linked to a coding sequence, even though they are not contiguous. Further, by way of example, a splice acceptor can be operably linked to a partial coding sequence if the splice acceptor enables delineation of an intron's 3′ boundary, and if translation of the resulting mature mRNA results in incorporation of the peptide sequence encoded by the partial coding sequence into the final protein product.

As used herein, the term “cleavage” refers to the breakage of the covalent backbone of a nucleic acid molecule. Cleavage can be initiated by a variety of methods including, but not limited to, enzymatic or chemical hydrolysis of a phosphodiester bond. Cleavage can refer to both a single-stranded nick and a double-stranded break. A double-stranded break can occur as a result of two distinct single-stranded nicks. Nucleic acid cleavage can result in the production of either blunt ends or staggered ends. In certain embodiments, rare-cutting endonucleases are used for targeted double-stranded or single-stranded DNA cleavage.

As used herein, the term “subject” can refer to an organism. The organism can include, but is not limited to, humans, non-human primates (e.g., macaques, chimpanzees), rodents (e.g., mice, rats, hamsters), livestock animals (e.g., cows, pigs, sheep, goats), companion animals (e.g., dogs, cats), aquatic species (e.g., zebrafish, salmon, trout), insects (e.g., Drosophila, mosquitoes, bees), birds (e.g., chickens, ducks), reptiles and amphibians (e.g., frogs, lizards), plants (e.g., Arabidopsis, crops such as corn, rice, wheat).

An “exogenous” molecule can refer to a small molecule (e.g., sugars, lipids, amino acids, fatty acids, phenolic compounds, alkaloids), or a macromolecule (e.g., protein, nucleic acid, carbohydrate, lipid, glycoprotein, lipoprotein, polysaccharide), or any modified derivative of the above molecules, or any complex comprising one or more of the above molecules, generated or present outside of a cell, or not normally present in a cell. Exogenous molecules can be introduced into cells. Methods for the introduction or “administering” of exogenous molecules into cells can include lipid-mediated transfer, electroporation, direct injection, cell fusion, particle bombardment, calcium phosphate co-precipitation, DEAE-dextran-mediated transfer and viral vector-mediated transfer. As defined herein, “administering” can refer to the delivery, the providing, or the introduction of exogenous molecules into a cell. If a transgene or a rare-cutting endonuclease is administered to a cell, then the transgene or rare-cutting endonuclease is delivered to, provided, or introduced into the cell. The rare-cutting endonuclease can be administered as purified protein, nucleic acid, or a mixture of purified protein and nucleic acid. The nucleic acid (i.e., RNA or DNA), can encode for the rare-cutting endonuclease, or a part of a rare-cutting endonuclease (e.g., a gRNA). The administering can be achieved though methods such as lipid-mediated transfer, electroporation, direct injection, cell fusion, particle bombardment, calcium phosphate co-precipitation, DEAE-dextran-mediated transfer, viral vector-mediated transfer, or any means suitable of delivering purified protein or nucleic acids, or a mixture of purified protein and nucleic acids, to a cell.

An “endogenous” molecule is a molecule that is present in a particular cell at a particular developmental stage under particular environmental conditions. An endogenous molecule can be a nucleic acid, a chromosome, the genome of a mitochondrion, chloroplast or other organelle, or a naturally occurring episomal nucleic acid. Additional endogenous molecules can include proteins, for example, transcription factors and enzymes.

As used herein, a “gene,” refers to a DNA region encoding that encodes a gene product, including all DNA regions which regulate the production of the gene product. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. As used herein, a “wild type gene” refers to a form of the gene that is present at the highest frequency in a particular population.

An “endogenous gene” refers to a DNA region normally present in a particular cell that encodes a gene product as well as all DNA regions which regulate the production of the gene product.

“Gene expression” refers to the conversion of the information, contained in a gene, into a gene product. A gene product can be the direct transcriptional product of a gene. For example, the gene product can be, but not limited to, mRNA, tRNA, rRNA, antisense RNA, ribozyme, structural RNA, or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Encoding” refers to the conversion of the information contained in a nucleic acid, into a product, wherein the product can result from the direct transcriptional product of a nucleic acid sequence. For example, the product can be, but not limited to, mRNA, tRNA, rRNA, antisense RNA, ribozyme, structural RNA, or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

A “target site” or “target sequence” defines a portion of a nucleic acid to which a rare-cutting endonuclease will bind, provided sufficient conditions for binding exist.

As used herein, the term “recombination” refers to a process of exchange of genetic information between two polynucleotides. The term “homologous recombination (HR)” refers to a specialized form of recombination that can take place, for example, during the repair of double-strand breaks. Homologous recombination requires nucleotide sequence homology present on a “donor” molecule. The donor molecule can be used by the cell as a template for repair of a double-strand break. Information within the donor molecule that differs from the genomic sequence at or near the double-strand break can be stably incorporated into the cell's genomic DNA.

The term “integrating” as used herein refers to the process of adding DNA to a target region of DNA. As described herein, integration can be facilitated by several different means, including non-homologous end joining, or homologous recombination. By way of example, integration of a user-supplied DNA molecule into a target gene can be facilitated by non-homologous end joining. Here, a targeted-double strand break is made within the target gene and a user-supplied DNA molecule is administered. The user-supplied DNA molecule can comprise exposed DNA ends to facilitate capture during repair of the target gene by non-homologous end joining. The exposed ends can be present on the DNA molecule upon administration (i.e., administration of a linear DNA molecule) or created upon administration to the cell (i.e., a rare-cutting endonuclease cleaves the user-supplied DNA molecule within the cell to expose the ends). Additionally, the user-supplied DNA molecule can be harbored on a viral vector, including an adeno-associated virus vector. In another example, integration occurs though homologous recombination. Here, the user-supplied DNA can harbor a left and right homology arm.

The term “transgene” as used herein refers to a sequence of nucleic acids that can be transferred to an organism or cell. The transgene may comprise a gene or sequence of nucleic acids not normally present in the target organism or cell. Additionally, the transgene may comprise a copy of a gene or sequence of nucleic acids that is normally present in the target organism or cell. A transgene can be an exogenous DNA sequence introduced into the cytoplasm or nucleus of a target cell.

As used herein, the term “insertion template” refers to a sequence of nucleic acids that can be integrated into the genome of an organism or cell. The insertion template may comprise a gene or sequence of nucleic acids not normally present in the target organism or cell. An insertion template can be an exogenous DNA sequence introduced into the cytoplasm or nucleus of a target cell.

As used herein, the term “pathogenic” refers to anything that can cause disease. A pathogenic mutation can refer to a modification in a gene which causes disease. A pathogenic gene refers to a gene comprising a modification which causes disease. By means of example, a pathogenic ATXN3 gene in patients with spinocerebellar ataxia 3 refers to an ATXN3 gene with an expanded CAG trinucleotide repeat, wherein the expanded CAG trinucleotide repeat causes the disease.

As used herein, the term “tail-to-tail” refers to an orientation of two units in opposite and reverse directions. The two units can be two sequences on a single nucleic acid molecule, where the 3′ end of each sequence are placed adjacent to each other. For example, a first nucleic acid having the elements, in a 5′ to 3′ direction, [splice acceptor 1]-[partial coding sequence 1]-[terminator 1] and a second nucleic acid having the elements [splice acceptor 2]-[partial coding sequence 2]-[terminator 2] can be placed in tail-to-tail orientation resulting in [splice acceptor 1]-[coding sequence 1]-[terminator 1]-[terminator 2 RC]-[coding sequence 2 RC]-[splice acceptor 2 RC], where RC refers to reverse complement.

The term “homologous” as used herein refers to a sequence of nucleic acids or amino acids having similarity to a second sequence of nucleic acids or amino acids. In some embodiments, the homologous sequences can have at least 80% sequence identity (e.g., 81%, 85%, 90%, 95%, 96%, 97%, 98%, or 99% sequence identity) to one another.

The term “coding sequence” as used herein refers to a sequence of nucleic acids that encodes a sequence of amino acids. The coding sequence can encode a sequence of amino acids that comprises all of the amino acids from a wild type or functional protein, or one or less amino acids as compared to the wild type protein or functional protein. The coding sequence can encode amino acids with homology to the wild type protein or functional protein.

The term “homology sequence” refers to a sequence of nucleic acids that comprises homology to a second nucleic acid. Homology sequence, for example, can be present on a donor molecule as an “arm of homology” or “homology arm.” A homology arm can be a sequence of nucleic acids within a donor molecule that facilitates homologous recombination with the second nucleic acid. As defined herein, a homology arm can also be referred to as an “arm”. In a donor molecule with two homology arms, the homology arms can be referred to as “arm 1” and “arm 2.” In one aspect, a cargo sequence can be flanked with first and second homology arm.

A 5′ or 3′ end of a nucleic acid molecule references the directionality and chemical orientation of the nucleic acid. As defined herein, the “5′ end of a gene” can comprise the exon with the start codon, but not the exon with the stop codon. As defined herein, the “3′ end of a gene” can comprise the exon with the stop codon, but not the exon with the start codon.

The percent sequence identity between a particular nucleic acid or amino acid sequence and a sequence referenced by a particular sequence identification number is determined as follows. First, a nucleic acid or amino acid sequence is compared to the sequence set forth in a particular sequence identification number using the BLAST 2 Sequences (B12seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the B12seq program can be found in the readme file accompanying BLASTZ. B12seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: −i is set to a file containing the first nucleic acid sequence to be compared (e.g., C:\seq1.txt); −j is set to a file containing the second nucleic acid sequence to be compared (e.g., C:\seq2.txt); −p is set to blastn; −o is set to any desired file name (e.g., C:\output.txt); −q is set to −1; −r is set to 2; and all other options are left at their default setting. For example, the following command can be used to generate an output file containing a comparison between two sequences: C:\B12seq −i c:\seq1.txt −j c:\seq2.txt −p blastn −o c:\output.txt −q −1 −r 2. To compare two amino acid sequences, the options of B12seq are set as follows: −i is set to a file containing the first amino acid sequence to be compared (e.g., C:\seq1.txt); −j is set to a file containing the second amino acid sequence to be compared (e.g., C:\seq2.txt); −p is set to blastp; −o is set to any desired file name (e.g., C:\output.txt); and all other options are left at their default setting. For example, the following command can be used to generate an output file containing a comparison between two amino acid sequences: C:\B12seq −i c:\seq1.txt −j c:\seq2.txt −p blastp −o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (e.g., 100 consecutive nucleotides or amino acid residues from a sequence set forth in an identified sequence), followed by multiplying the resulting value by 100. The percent sequence identity value is rounded to the nearest tenth.

As described here, “step 1” refers to the process of administering a rare-cutting endonuclease and an insertion template to a population of cells. The rare-cutting endonuclease can bind to and cleave a “target site 1” within the genome of a cell.

As described here, “step 2” refers to the process that occurs after step 1. Step 2 includes administering a rare-cutting endonuclease and an insertion template to the same population of cells as in step 1. The rare-cutting endonuclease can bind to and cleave a “target site 2” within the genome of a cell.

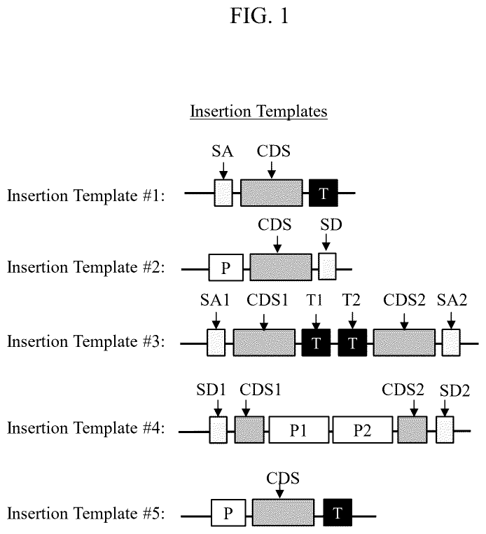

In one embodiment, this document features materials and methods increasing the frequency of insertion events within a population of cells. The materials and methods include the use of insertion templates (FIG. 1). In one embodiment, the insertion template can comprise a nucleic acid sequence comprising, from 5′ to 3′, a splice acceptor, a coding sequence and a terminator. This insertion template can be integrated within an intron of an endogenous gene. In another embodiment, the insertion templates can comprise a nucleic acid sequence comprising, from 5′ to 3′, a promoter, a coding sequence, and a splice donor. This insertion template can be integrated within an intron of an endogenous gene. In another embodiment, the insertion templates can be a bidirectional insertion template and can comprise a nucleic acid sequence comprising, from 5′ to 3′, a first splice acceptor, a first coding sequence, a first terminator, a second terminator reverse complement, a second coding sequence reverse complement, and a second splice acceptor reverse complement. This insertion template can be integrated within an intron of an endogenous gene. In another embodiment, the insertion templates can comprise a nucleic acid sequence comprising, from 5′ to 3′, a first splice donor reverse complement, a first coding sequence reverse complement, a first promoter reverse complement, a second promoter, a second coding sequence and a second splice donor. This insertion template can be integrated within an intron of an endogenous gene. In another embodiment, the insertion templates can comprise a nucleic acid sequence comprising, from 5′ to 3′, a promoter, a coding sequence and a terminator. This insertion template can be integrated within an intergenic region within the genome of a cell, or within an intron of an endogenous gene.

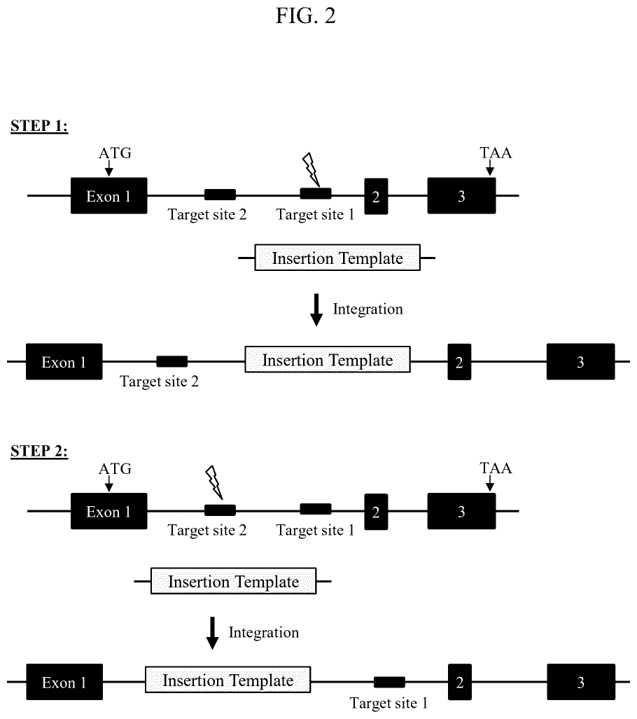

In one embodiment, this document features a two-step process for increasing the number of gene insertion events within a population of cells, wherein the gene insertion event occurs within an intron of an endogenous gene (FIG. 2). For step 1, an insertion template and a rare-cutting endonuclease are administered to the population of cells. The rare-cutting endonuclease creates a targeted double-strand break within a first target site (target site 1), which facilitates integration of the insertion template. For step 2, the same population of cells is subsequently administered the insertion template and a rare-cutting endonuclease that creates a targeted double-strand break within a second target site (target site 2).

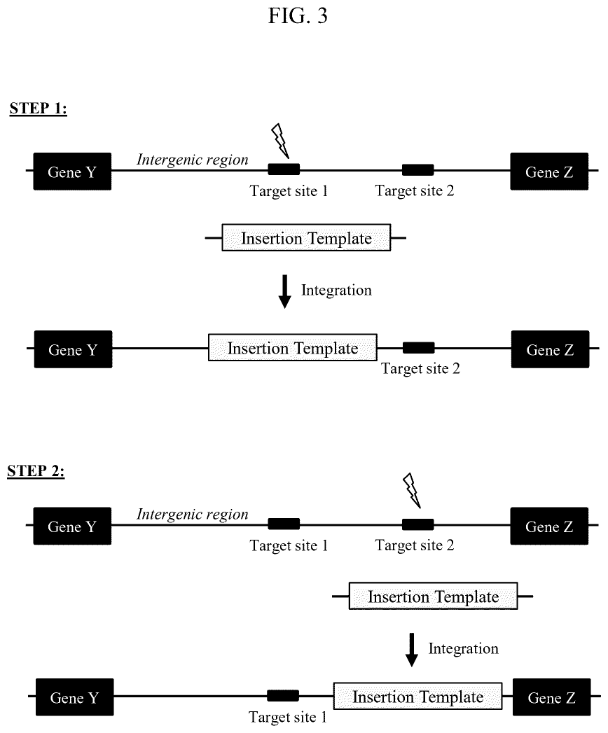

In another embodiment, this document features a two-step process for increasing the number of gene insertion events within a population of cells, wherein the gene insertion event occurs within an intergenic region of the genome (FIG. 3). For step 1, an insertion template and a rare-cutting endonuclease is administered to the population of cells. The rare-cutting endonuclease creates a targeted double-strand break within a first target site (target site 1), which facilitates integration of the insertion template. For step 2, the same population of cells is subsequently administered the insertion template and a rare-cutting endonuclease that creates a targeted double-strand break within a second target site (target site 2). In a preferred embodiment, the insertion template used here comprises, from 5′ to 3′, a promoter, coding sequence and terminator. The intergenic region can be any region between genes that is suitable for gene expression.

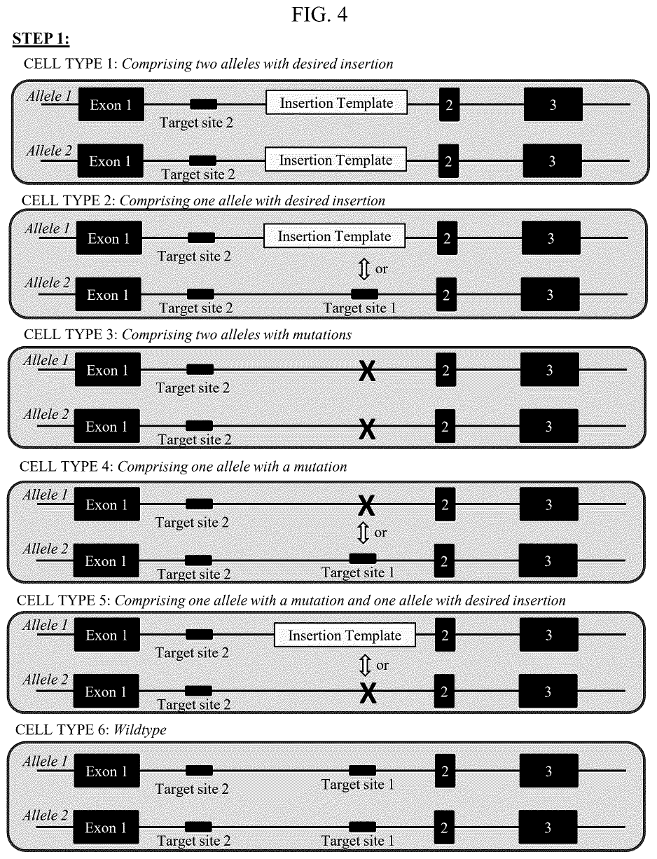

In one embodiment, the methods presented herein provide a means for increasing the number of gene insertion events within a population of cells. Following step 1, only a fraction of the population of cells will comprise an insertion template into target site 1 (FIG. 4). There are six different cell types within the population of cells after step 1. These cell types have differing combinations of insertion, mutation, or wild type sequences at target site 1 for the different alleles. Cell type 1 comprises two insertion events in allele 1 and allele 2. Cell type 2 comprises a single insertion event within one allele, while the other allele has a wild type target site 1. Cell type 3 comprises two knockout mutations that destroy target site 1 on both alleles. Cell type 4 comprises a single knockout mutation in one allele, where the other allele is wild type. Cell type 5 comprises a single insertion event in one allele, where the other allele has a knockout mutation that destroys target site 1. Cell type 6 comprises no gene editing events, and both alleles remain wild type. In step 1, three of the six different cell types have potential to produce a coding sequence of interest.

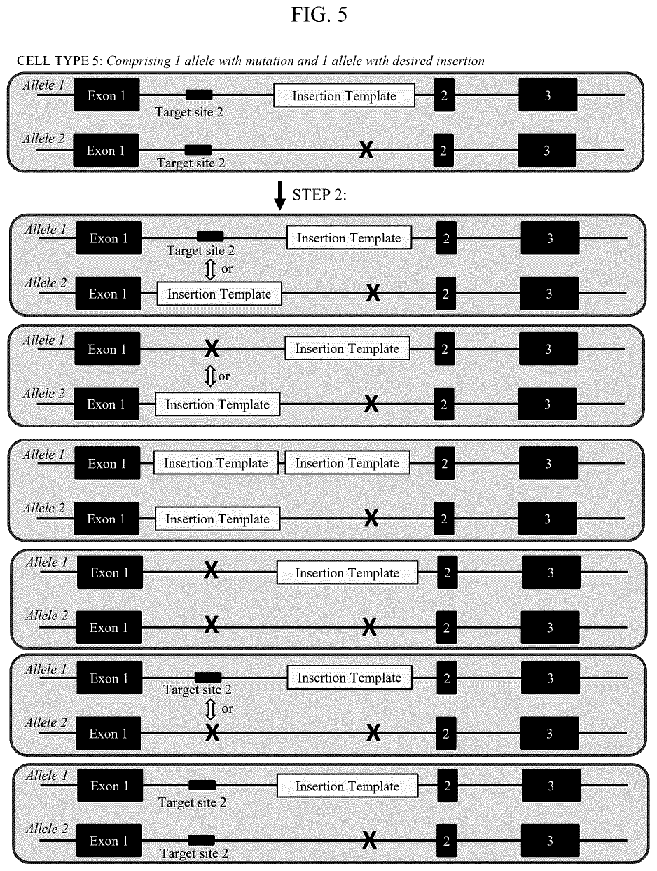

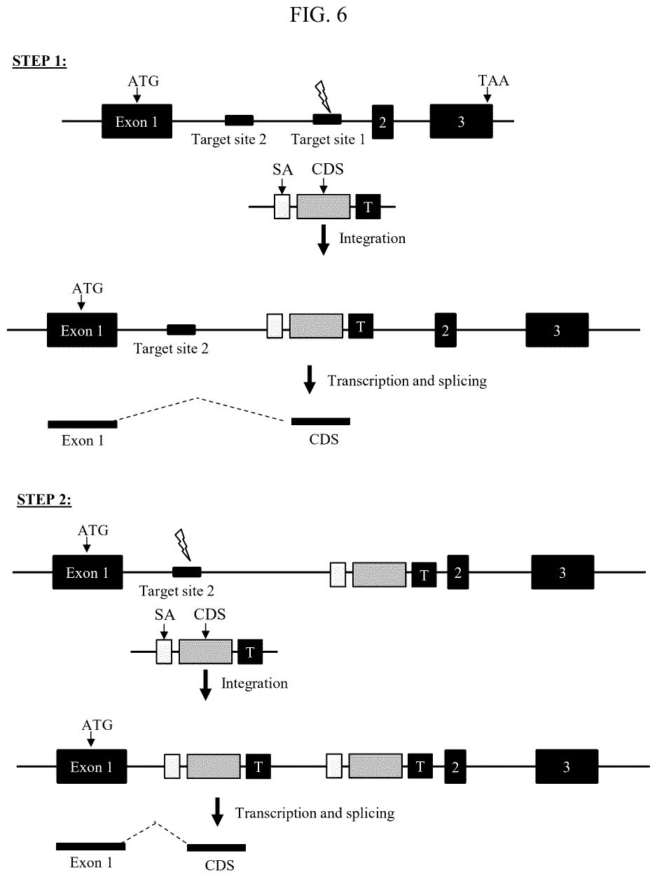

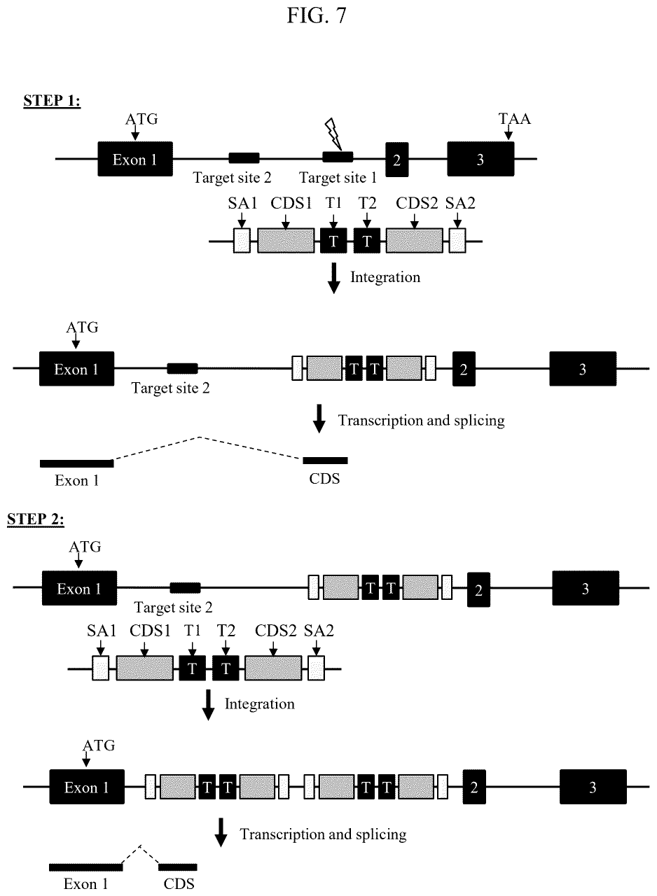

In another embodiment, step 1 is followed by step 2, which comprises administering the same population of cells with the insertion template along with a rare-cutting endonuclease that targets a second target site. An example of the subsequence outcomes produced from editing cell type 5 is shown in FIG. 5. Notably, in step 1, three of the six different cell types have potential to produce a coding sequence of interest; however, redosing with the same rare-cutting endonuclease and insertion template composition can only result in an additional benefit from three of the six cell types. In other words, three of the six cell types are incapable of acquiring a desired gene insertion event and cannot contribute to increasing the overall insertion levels of the final cell population. By using a second rare-cutting endonuclease, target a second site, all cell types are capable of acquiring a desired gene insertion event, which will significantly increase the number of cells with the desired insertion template. In some instances, two insertion events will occur in a single allele; however, the design of the insertion templates (FIG. 1) permit expression of the desired coding sequence, regardless of the number of insertion templates within the target site (FIG. 6 and FIG. 7).

In one embodiment, the insertion templates can comprise coding sequences operably linked to a splice donor or splice acceptor. When the insertion template is integrated into an endogenous gene the coding sequence is transcribed into mRNA by the endogenous gene's promoter. The coding sequence can be designed to correct defective coding sequences, introduce mutations, or introduce novel peptide sequences. The coding sequence can encode purification tags (e.g., glutathione-S-transferase, poly(His), maltose binding protein, Strep-tag, Myc-tag, AviTag, HA-tag, or chitin binding protein) or reporter proteins (e.g., GFP, RFP, lacZ, cat, luciferase, puro, neomycin). The coding sequence can encode wild type or functional versions of endogenous genes.

In another embodiment the coding sequence can be within a bidirectional template, where there is a first and second coding sequence in bidirectional orientation. The first and second coding sequences are positioned within the insertion template in opposite directions (i.e., in tail-to-tail orientations). When the insertion template is integrated into an endogenous gene in forward or reverse directions, the first or second coding sequence is transcribed into mRNA by the endogenous gene's promoter. The coding sequences can be designed to correct defective coding sequences, introduce mutations, or introduce novel peptide sequences. The first and second coding sequence can be the same nucleic acid sequence and code for the same protein. Alternatively, the first and second coding sequence can be different nucleic acid sequences and code for the same protein (i.e., using the degeneracy of codons). The coding sequence can encode purification tags (e.g., glutathione-S-transferase, poly(His), maltose binding protein, Strep-tag, Myc-tag, AviTag, HA-tag, or chitin binding protein) or reporter proteins (e.g., GFP, RFP, lacZ, cat, luciferase, puro, neomycin).

In one embodiment, the coding sequences can be designed to carry amino acids that are encoded by endogenous genes, including fibrinogen, prothrombin, tissue factor, Factor V, Factor VII, Factor VIII, Factor IX, Factor X, Factor XI, Factor XII (Hageman factor), Factor XIII (fibrin-stabilizing factor), von Willebrand factor, prekallikrein, high molecular weight kininogen (Fitzgerald factor), fibronectin, antithrombin III, heparin cofactor II, protein C, protein S, protein Z, protein Z-related protease inhibitor, plasminogen, alpha 2-antiplasmin, tissue plasminogen activator, urokinase, plasminogen activator inhibitor-1, plasminogen activator inhibitor-2, glucocerebrosidase (GBA), α-galactosidase A (GLA), iduronate sulfatase (IDS), iduronidase (IDUA), acid sphingomyelinase (SMPD1), MMAA, MMAB, MMACHC, MMADHC (C2orf25), MTRR, LMBRD1, MTR, propionyl-CoA carboxylase (PCC) (PCCA and/or PCCB subunits), a glucose-6-phosphate transporter (G6PT) protein or glucose-6-phosphatase (G6Pase), an LDL receptor (LDLR), ApoB, LDLRAP-1, a PCSK9, a mitochondrial protein such as NAGS (N-acetylglutamate synthetase), CPS1 (carbamoyl phosphate synthetase I), and OTC (ornithine transcarbamylase), ASS (argininosuccinic acid synthetase), ASL (argininosuccinase acid lyase) and/or ARGI (arginase), and/or a solute carrier family 25 (SLC25A13, an aspartate/glutamate carrier) protein, UGT1A1 or UDP glucuronsyltransferase polypeptide A1, fumarylacetoacetate hydrolyase (FAH), alanine-glyoxylate aminotransferase (AGXT), a glyoxylate reductase/hydroxypyruvate reductase (GRHPR) protein, a transthyretin gene (TTR) protein, ATP7B, phenylalanine hydroxylase (PAH), USH2A, ATXN, and lipoprotein lyase (LPL).

In one embodiment, the coding sequence can encode amino acid sequence from functional proteins that are related to the genetic disease including achondroplasia, achromatopsia, acid maltase deficiency, adenosine deaminase deficiency, adrenoleukodystrophy, aicardi syndrome, alpha-1 antitrypsin deficiency, alpha-thalassemia, androgen insensitivity syndrome, pert syndrome, arrhythmogenic right ventricular dysplasia, ataxia telangictasia, barth syndrome, beta-thalassemia, blue rubber bleb nevus syndrome, canavan disease, chronic granulomatous diseases (CGD), cri du chat syndrome, cystic fibrosis, dercum's disease, ectodermal dysplasia, fanconi anemia, fibrodysplasia ossificans progressive, fragile X syndrome, galactosemis, Gaucher's disease, generalized gangliosidoses (e.g., GM1), hemochromatosis, the hemoglobin C mutation in the 6th codon of beta-globin (HbC), hemophilia, Huntington's disease, Hurler Syndrome, hypophosphatasia, Klinefleter syndrome, Krabbes Disease, Langer-Giedion Syndrome, leukocyte adhesion deficiency, leukodystrophy, long QT syndrome, Marfan syndrome, Moebius syndrome, mucopolysaccharidosis (MPS), nail patella syndrome, nephrogenic diabetes insipdius, neurofibromatosis, Neimann-Pick disease, osteogenesis imperfecta, porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, Wiskott-Aldrich syndrome, X-linked lymphoproliferative syndrome, lysosomal storage diseases (e.g., Gaucher's disease, GM1, Fabry disease and Tay-Sachs disease), mucopolysaccahidosis (e.g. Hunter's disease, Hurler's disease), hemoglobinopathies (e.g., sickle cell diseases, HbC, α-thalassemia, β-thalassemia) and hemophilias.

In one embodiment, the methods and insertion templates provided herein can be used to express therapeutic coding sequences. The therapeutic coding sequence can be designed to treat the symptoms of the genetic diseases, including achondroplasia, achromatopsia, acid maltase deficiency, adenosine deaminase deficiency, adrenoleukodystrophy, aicardi syndrome, alpha-1 antitrypsin deficiency, alpha-thalassemia, androgen insensitivity syndrome, pert syndrome, arrhythmogenic right ventricular dysplasia, ataxia telangictasia, barth syndrome, beta-thalassemia, blue rubber bleb nevus syndrome, canavan disease, chronic granulomatous diseases (CGD), cri du chat syndrome, cystic fibrosis, dercum's disease, ectodermal dysplasia, fanconi anemia, fibrodysplasia ossificans progressive, fragile X syndrome, galactosemis, Gaucher's disease, generalized gangliosidoses (e.g., GM1), hemochromatosis, the hemoglobin C mutation in the 6th codon of beta-globin (HbC), hemophilia, Huntington's disease, Hurler Syndrome, hypophosphatasia, Klinefleter syndrome, Krabbes Disease, Langer-Giedion Syndrome, leukocyte adhesion deficiency, leukodystrophy, long QT syndrome, Marfan syndrome, Moebius syndrome, mucopolysaccharidosis (MPS), nail patella syndrome, nephrogenic diabetes insipdius, neurofibromatosis, Neimann-Pick disease, osteogenesis imperfecta, porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, Wiskott-Aldrich syndrome, X-linked lymphoproliferative syndrome, lysosomal storage diseases (e.g., Gaucher's disease, GM1, Fabry disease and Tay-Sachs disease), mucopolysaccahidosis (e.g. Hunter's disease, Hurler's disease), hemoglobinopathies (e.g., sickle cell diseases, HbC, α-thalassemia, β-thalassemia) and hemophilias.

As described herein, the insertion templates can be in a viral or non-viral vector. The vectors can be in the form of circular or linear double-stranded or single stranded DNA. The insertion template can be conjugated or associated with a reagent that facilitates stability or cellular update. The reagent can be lipids (e.g., lipid nanoparticles), calcium phosphate, cationic polymers, DEAE-dextran, dendrimers, polyethylene glycol (PEG) cell penetrating peptides, gas-encapsulated microbubbles or magnetic beads. The insertion template can be incorporated into a viral particle. The virus can be retroviral, adenoviral, adeno-associated vectors (AAV), herpes simplex, pox virus, hybrid adenoviral vector, epstein-bar virus, lentivirus, or herpes simplex virus.

In certain embodiments, the AAV vectors as described herein can be derived from any AAV. In certain embodiments, the AAV vector is derived from the defective and nonpathogenic parvovirus adeno-associated type 2 virus. All such vectors are derived from a plasmid that retains only the AAV 145 bp inverted terminal repeats flanking the insertion template. Efficient gene transfer and stable insertion template delivery due to integration into the genomes of the transduced cell are key features for this vector system. (Wagner et al., Lancet 351:9117 1702-3, 1998; Kearns et al., Gene Ther. 9:748-55, 1996). Other AAV serotypes, including AAV1, AAV2, AAV3, AAV4, AAV5, AAV6, AAV7, AAV8, AAV9 and AAVrh.10 and any novel AAV serotype can also be used in accordance with the present invention. In some embodiments, chimeric AAV is used where the viral origins of the long terminal repeat (LTR) sequences of the viral nucleic acid are heterologous to the viral origin of the capsid sequences. Non-limiting examples include chimeric virus with LTRs derived from AAV2 and capsids derived from AAV5, AAV6, AAV8 or AAV9 (i.e. AAV2/5, AAV2/6, AAV2/8 and AAV2/9, respectively).

In one embodiment, the first and second target sites are within an intron of an endogenous gene. The second target site can be either upstream or downstream of the first target site. The second target site can be within the same intron as the first target site. The second target site can be a certain number of nucleotides away from the first target site. The second target site can be 1 or more, 10 or more, 20 or more, 30 or more, 40 or more, 50 or more, 100 or more, or 1000 or more nucleotides away from the first target site. ‘

In one embodiment, step 2 takes place at a certain time after step 1. The time between step 1 and step 2 can be 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 91 days, 92 days, 93 days, 94 days, 95 days, 96 days, 97 days, 98 days, 99 days, 100 days, 1 month, 2 months, 3 months, 4 months, 5 months, 6 months, 7 months, 8 months, 9 months, 10 months, 11 months, 12 months, 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, or 20 years.

In one embodiment, the composition administered in step 2 comprises the same insertion template as in step 1. In another embodiment, the composition administered in step 2 comprises a different insertion template with different nucleic acid sequences than the insertion template used in step 1; however, the function of the insertion template in step 2 is the same as in step 1. To illustrate by means of example, the insertion template used in step 2 can comprise different homology arm sequences but harbor the same coding sequence. In another example, the insertion template used in step 2 can comprise different splice acceptor sequences, but the splice acceptor sequences still perform the same function as the splice acceptor sequences within the insertion template used in step 1. In another example, the insertion template used in step 2 can comprise different coding sequences, where the coding sequence differs in nucleic acid sequence, but still encodes the same amino acid sequence as the coding sequence within the insertion template used in step 1. In all cases within these examples, the insertion template used in step 2 carries out the same function as the insertion template used in step 1, and results in expression of the same coding-sequence of interest.

In a specific embodiment, the insertion template can comprise an insertion template with a splice acceptor, a coding sequence encoding amino acids from the alpha-1 antitrypsin protein, and a terminator. The insertion template can be integrated within intron 1 of the albumin gene. Both target site 1 and target site 2 can be within intron 1 of the albumin gene. In one embodiment, target site 1 comprises the nucleic acid sequence shown in SEQ ID NO: 6.

In a specific embodiment, the insertion template can comprise an insertion template with a splice acceptor, a coding sequence encoding amino acids from the factor 9 protein, and a terminator. The insertion template can be integrated within intron 1 of the albumin gene. Both target site 1 and target site 2 can be within intron 1 of the albumin gene. In one embodiment, target site 1 comprises the nucleic acid sequence shown in SEQ ID NO: 6.

The methods and compositions described herein are applicable to any eukaryotic organism in which it is desired to alter the organism through genomic modification. The eukaryotic organisms include plants, algae, animals, fungi and protists. The eukaryotic organisms can also include plant cells, algae cells, animal cells, fungal cells and protist cells.

Exemplary mammalian cells include, but are not limited to, oocytes, K562 cells, CHO (Chinese hamster ovary) cells, HEP-G2 cells, BaF-3 cells, Schneider cells, COS cells (monkey kidney cells expressing SV40 T-antigen), CV-1 cells, HuTu80 cells, NTERA2 cells, NB4 cells, HL-60 cells and HeLa cells, 293 cells (see, e.g., Graham et al. (1977) J. Gen. Virol. 36:59), and myeloma cells like SP2 or NS0 (see, e.g., Galfre and Milstein (1981) Meth. Enzymol. 73 (B): 3 46). Peripheral blood mononucleocytes (PBMCs) or T-cells can also be used, as can embryonic and adult stem cells. For example, stem cells that can be used include embryonic stem cells (ES), induced pluripotent stem cells (iPSC), mesenchymal stem cells, hematopoietic stem cells, liver stem cells, skin stem cells and neuronal stem cells.

The methods and compositions of the invention can be used in the production of modified organisms. The modified organisms can be small mammals, companion animals, livestock, and primates. Non-limiting examples of rodents may include mice, rats, hamsters, gerbils, and guinea pigs. Non-limiting examples of companion animals may include cats, dogs, rabbits, hedgehogs, and ferrets. Non-limiting examples of livestock may include horses, goats, sheep, swine, llamas, alpacas, and cattle. Non-limiting examples of primates may include capuchin monkeys, chimpanzees, lemurs, macaques, marmosets, tamarins, spider monkeys, squirrel monkeys, and vervet monkeys. The methods and compositions of the invention can be used in humans.

Exemplary plants and plant cells which can be modified using the methods described herein include, but are not limited to, monocotyledonous plants (e.g., wheat, maize, rice, millet, barley, sugarcane), dicotyledonous plants (e.g., soybean, potato, tomato, alfalfa), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); vegetative crops for consumption (e.g. soybean and other legumes, squash, peppers, eggplant, celery etc), flowering plants (e.g., petunia, rose, chrysanthemum), conifers and pine trees (e.g., pine fir, spruce); poplar trees (e.g. P. tremula×P. alba); fiber crops (cotton, jute, flax, bamboo) plants used in phytoremediation (e.g., heavy metal accumulating plants); oil crops (e.g., sunflower, rape seed) and plants used for experimental purposes (e.g., Arabidopsis). The methods disclosed herein can be used within the genera Asparagus, Avena, Brassica, Citrus, Citrullus, Capsicum, Cucurbita, Daucus, Erigeron, Glycine, Gossypium, Hordeum, Lactuca, Lolium, Lycopersicon, Malus, Manihot, Nicotiana, Orychophragmus, Oryza, Persea, Phaseolus, Pisum, Pyrus, Prunus, Raphanus, Secale, Solanum, Sorghum, Triticum, Vitis, Vigna, and Zea. The term plant cells include isolated plant cells as well as whole plants or portions of whole plants such as seeds, callus, leaves, and roots. The present disclosure also encompasses seeds of the plants described above wherein the seed has the has been modified using the compositions and/or methods described herein. The present disclosure further encompasses the progeny, clones, cell lines or cells of the transgenic plants described above wherein said progeny, clone, cell line or cell has the insertion template or gene construct. Exemplary algae species include microalgae, diatoms, Botryococcus braunii, Chlorella, Dunaliella tertiolecta, Gracileria, Pleurochrysis carterae, Sorgassum and Ulva.

The methods described in this document can include the use of rare-cutting endonucleases for stimulating homologous recombination or non-homologous integration of an insertion template molecule into an endogenous gene. The rare-cutting endonuclease can include CRISPR, TALENs, or zinc-finger nucleases (ZFNs). The CRISPR system can include CRISPR/Cas9 or CRISPR/Cas12a (Cpf1). The CRISPR system can include variants which display broad PAM capability (Hu et al., Nature 556, 57-63, 2018; Nishimasu et al., Science DOI: 10.1126, 2018) or higher on-target binding or cleavage activity (Kleinstiver et al., Nature 529:490-495, 2016). The gene editing reagent can be in the format of a nuclease (Mali et al., Science 339:823-826, 2013; Christian et al., Genetics 186:757-761, 2010), nickase (Cong et al., Science 339:819-823, 2013; Wu et al., Biochemical and Biophysical Research Communications 1:261-266, 2014), CRISPR-FokI dimers (Tsai et al., Nature Biotechnology 32:569-576, 2014), or paired CRISPR nickases (Ran et al., Cell 154:1380-1389, 2013).

Genome or gene editing generally refers to the process of modifying the nucleotide sequence of a genome, preferably in a precise or pre-determined manner. Examples of methods of genome editing described herein include methods of using rare-cutting endonucleases to cut deoxyribonucleic acid (DNA) at precise target locations in the genome, thereby creating single-strand or double-strand DNA breaks at particular locations within the genome. Such breaks can be and regularly are repaired by natural, endogenous cellular processes, such as homology-directed repair (HR) and non-homologous end joining (NHEJ), as recently reviewed in Cox et al., Nature Medicine 21 (2), 121-31 (2015). These two main DNA repair processes consist of a family of alternative pathways. NHEJ directly joins the DNA ends resulting from a double-strand break, sometimes with the loss or addition of nucleotide sequence, which may disrupt or enhance gene expression. HR utilizes a homologous sequence, or donor sequence, as a template for inserting a defined DNA sequence at the break point. The homologous sequence can be in the endogenous genome, such as a sister chromatid. Alternatively, the donor can be an exogenous nucleic acid, such as a plasmid, a single-strand oligonucleotide, a double-stranded oligonucleotide, a duplex oligonucleotide or a virus, that has regions of high homology with the nuclease-cleaved locus, but which can also contain additional sequence or sequence changes including deletions that can be incorporated into the cleaved target locus. A third repair mechanism can be microhomology-mediated end joining (MMEJ), also referred to as “Alternative NHEJ,” in which the genetic outcome is similar to NHEJ in that small deletions and insertions can occur at the cleavage site. MMEJ can make use of homologous sequences of a few basepairs flanking the DNA break site to drive a more favored DNA end joining repair outcome, and recent reports have further elucidated the molecular mechanism of this process; see, e.g., Cho and Greenberg, Nature 518, 174-76 (2015); Kent et al., Nature Structural and Molecular Biology, Adv. Online doi: 10.1038/nsmb.2961 (2015); Mateos-Gomez et al., Nature 518, 254-57 (2015); Ceccaldi et al., Nature 528, 258-62 (2015).

CRISPR (Clustered Regularly Interspaced Short Palindromic Repeats) genomic locus can be found in the genomes of many prokaryotes (e.g., bacteria and archaea). In prokaryotes, the CRISPR locus encodes products that function as a type of immune system to help defend the prokaryotes against foreign invaders, such as virus and phage. There are three stages of CRISPR locus function: integration of new sequences into the CRISPR locus, expression of CRISPR RNA (crRNA), and silencing of foreign invader nucleic acid. Five types of CRISPR systems (e.g., Type I, Type II, Type III, Type U, and Type V) have been identified.

A CRISPR locus includes a number of short repeating sequences referred to as “repeats.” When expressed, the repeats can form secondary structures (e.g., hairpins) and/or comprise unstructured single-stranded sequences. The repeats usually occur in clusters and frequently diverge between species. The repeats are regularly interspaced with unique intervening sequences referred to as “spacers,” resulting in a repeat-spacer-repeat locus architecture. The spacers are identical to or have high homology with known foreign invader sequences. A spacer-repeat unit encodes a crisprRNA (crRNA), which is processed into a mature form of the spacer-repeat unit. A crRNA comprises a “seed” or spacer sequence that is involved in targeting a target nucleic acid (in the naturally occurring form in prokaryotes, the spacer sequence targets the foreign invader nucleic acid). A spacer sequence is located at the 5′ or 3′ end of the crRNA.

A CRISPR locus also comprises polynucleotide sequences encoding CRISPR Associated (Cas) genes. Cas genes encode endonucleases involved in the biogenesis and the interference stages of crRNA function in prokaryotes. Some Cas genes comprise homologous secondary and/or tertiary structures.

crRNA biogenesis in a Type II CRISPR system in nature requires a trans-activating CRISPR RNA (tracrRNA). The tracrRNA can be modified by endogenous RNaseIII, and then hybridizes to a crRNA repeat in the pre-crRNA array. Endogenous RNaseIII can be recruited to cleave the pre-crRNA. Cleaved crRNAs can be subjected to exoribonuclease trimming to produce the mature crRNA form (e.g., 5′ trimming). The tracrRNA can remain hybridized to the crRNA, and the tracrRNA and the crRNA associate with a rare-cutting endonuclease (e.g., Cas9). The crRNA of the crRNA-tracrRNA-Cas9 complex can guide the complex to a target nucleic acid to which the crRNA can hybridize. Hybridization of the crRNA to the target nucleic acid can activate Cas9 for targeted nucleic acid cleavage. The target nucleic acid in a Type II CRISPR system is referred to as a protospacer adjacent motif (PAM). In nature, the PAM is essential to facilitate binding of a rare-cutting endonuclease (e.g., Cas9) to the target nucleic acid. Type II systems (also referred to as Nmeni or CASS4) are further subdivided into Type II-A (CASS4) and II-B (CASS4a). Jinek et al., Science, 337 (6096): 816-821 (2012) showed that the CRISPR/Cas9 system is useful for RNA-programmable genome editing, and international patent application publication number WO2013/176772 provides numerous examples and applications of the CRISPR/Cas endonuclease system for site-specific gene editing.

Type V CRISPR systems have several important differences from Type II systems. For example, Cpf1 is a single RNA-guided endonuclease that, in contrast to Type II systems, lacks tracrRNA. In fact, Cpf1-associated CRISPR arrays can be processed into mature crRNAs without the requirement of an additional trans-activating tracrRNA. The Type V CRISPR array can be processed into short mature crRNAs of 42-44 nucleotides in length, with each mature crRNA beginning with 19 nucleotides of direct repeat followed by 23-25 nucleotides of spacer sequence. In contrast, mature crRNAs in Type II systems can start with 20-24 nucleotides of spacer sequence followed by about 22 nucleotides of direct repeat. Also, Cpf1 can utilize a T-rich protospacer-adjacent motif such that Cpf1-crRNA complexes efficiently cleave target DNA preceded by a short T-rich PAM, which is in contrast to the G-rich PAM following the target DNA for Type II systems. Thus, Type V systems cleave at a point that is distant from the PAM, while Type II systems cleave at a point that is adjacent to the PAM. In addition, in contrast to Type II systems, Cpf1 cleaves DNA via a staggered DNA double-stranded break with a 4 or 5 nucleotide 5′ overhang. Type II systems cleave via a blunt double-stranded break. Similar to Type II systems, Cpf1 contains a predicted RuvC-like endonuclease domain, but lacks a second HNH endonuclease domain, which is in contrast to Type II systems.

Exemplary CRISPR/Cas polypeptides include the Cas9 polypeptides as published in Fonfara et al., Nucleic Acids Research, 42:2577-2590 (2014). Fonfara et al., also provides PAM sequences for the Cas9 polypeptides from various species.

A rare-cutting endonuclease is a nuclease used in genome editing to cleave DNA. The rare-cutting endonuclease can be administered to a cell or a patient as either: one or more polypeptides, or one or more mRNAs encoding the polypeptide. In the context of a CRISPR/Cas9 or CRISPR/Cpf1 system, the rare-cutting endonuclease can bind to a guide RNA that, in turn, specifies the site in the target DNA to which the polypeptide is directed. In the CRISPR/Cas9 or CRISPR/Cpf1 systems disclosed herein, the rare-cutting endonuclease can be an endonuclease, such as a DNA endonuclease.

A rare-cutting endonuclease can comprise a plurality of nucleic acid-cleaving (i.e., nuclease) domains. Two or more nucleic acid-cleaving domains can be linked together via a linker. For example, the linker can comprise a flexible linker. Linkers can comprise 1, 2, 3, 4, 5, 6, 7, 8, 9, 10, 11, 12, 13, 14, 15, 16, 17, 18, 19, 20, 21, 22, 23, 24, 25, 30, 35, 40 or more amino acids in length.

Naturally-occurring wild-type Cas9 enzymes comprise two nuclease domains, a HNH nuclease domain and a RuvC domain. Herein, the term “Cas9” refers to both a naturally-occurring and a recombinant Cas9. Cas9 enzymes contemplated herein can comprise a HNH or HNH-like nuclease domain, and/or a RuvC or RuvC-like nuclease domain.

HNH or HNH-like domains comprise a McrA-like fold. HNH or HNH-like domains comprises two antiparallel β-strands and an α-helix. HNH or HNH-like domains comprises a metal binding site (e.g., a divalent cation binding site). HNH or HNH-like domains can cleave one strand of a target nucleic acid (e.g., the complementary strand of the crRNA targeted strand).

RuvC or RuvC-like domains comprise an RNaseH or RNaseH-like fold. RuvC/RNaseH domains are involved in a diverse set of nucleic acid-based functions including acting on both RNA and DNA. The RNaseH domain comprises 5 β-strands surrounded by a plurality of α-helices. RuvC/RNaseH or RuvC/RNaseH-like domains comprise a metal binding site (e.g., a divalent cation binding site). RuvC/RNaseH or RuvC/RNaseH-like domains can cleave one strand of a target nucleic acid (e.g., the non-complementary strand of a double-stranded target DNA).

Rare-cutting endonucleases can introduce double-strand breaks or single-strand breaks in nucleic acids, e.g., genomic DNA. The double-strand break can stimulate a cell's endogenous DNA-repair pathways (e.g., homology-dependent repair (HR) or NHEJ or alternative non-homologous end joining (A-NHEJ) or microhomology-mediated end joining (MMEJ)). NHEJ can repair cleaved target nucleic acid without the need for a homologous template. This can sometimes result in small deletions or insertions (indels) in the target nucleic acid at the site of cleavage, and can lead to disruption or alteration of gene expression. NHEJ can also capture exogenously-supplied insertion templates and result in direct insertion of DNA into the site of cleavage. HR can occur when a homologous repair template, or donor, is available. The homologous donor template can comprise sequences that are homologous to sequences flanking the target nucleic acid cleavage site. The sister chromatid can be used by the cell as the repair template. However, for the purposes of genome editing, the repair template can be supplied as an exogenous nucleic acid, such as a plasmid, duplex oligonucleotide, single-strand oligonucleotide or viral nucleic acid. With exogenous donor templates, an additional nucleic acid sequence (such as an insertion template) or modification (such as a single or multiple base change or a deletion) can be introduced between the flanking regions of homology so that the additional or altered nucleic acid sequence also becomes incorporated into the target locus. MMEJ can result in a genetic outcome that is similar to NHEJ in that small deletions and insertions can occur at the cleavage site. MMEJ can make use of homologous sequences of a few basepairs flanking the cleavage site to drive a favored end-joining DNA repair outcome. In some instances, it may be possible to predict likely repair outcomes based on analysis of potential microhomologies in the nuclease target regions.

Non-limiting examples of Cas9 orthologs from other bacterial strains include but are not limited to, Cas proteins identified in Acaryochloris marina MBIC11017; Acetohalobium arabaticum DSM 5501; Acidithiobacillus caldus; Acidithiobacillus ferrooxidans ATCC 23270; Alicyclobacillus acidocaldarius LAA1; Alicyclobacillus acidocaldarius subsp. acidocaldarius DSM 446; Allochromatium vinosum DSM 180; Ammonifex degensii KC4; Anabaena variabilis ATCC 29413; Arthrospira maxima CS-328; Arthrospira platensis str. Paraca; Arthrospira sp. PCC 8005; Bacillus pseudomycoides DSM 12442; Bacillus selenitireducens MLS10; Burkholderiales bacterium 1_1_47; Caldicelulosiruptor becscii DSM 6725; Candidatus Desulforudis audaxviator MP104C; Caldicellulosiruptor hydrothermalis_108; Clostridium phage c-st; Clostridium botulinum A3 str. Loch Maree; Clostridium botulinum Ba4 str. 657; Clostridium difficile QCD-63q42; Crocosphaera watsonii WH 8501; Cyanothece sp. ATCC 51142; Cyanothece sp. CCY0110; Cyanothece sp. PCC 7424; Cyanothece sp. PCC 7822; Exiguobacterium sibiricum 255-15; Finegoldia magna ATCC 29328; Ktedonobacter racemifer DSM 44963; Lactobacillus delbrueckii subsp. bulgaricus PB2003/044-T3-4; Lactobacillus salivarius ATCC 11741; Listeria innocua; Lyngbya sp. PCC 8106; Marinobacter sp. ELB17; Methanohalobium evestigatum Z-7303; Microcystis phage Ma-LMM01; Microcystis aeruginosa NIES-843; Microscilla marina ATCC 23134; Microcoleus chthonoplastes PCC 7420; Neisseria meningitidis; Nitrosococcus halophilus Nc4; Nocardiopsis dassonvillei subsp. dassonvillei DSM 43111; Nodularia spumigena CCY9414; Nostoc sp. PCC 7120; Oscillatoria sp. PCC 6506; Pelotomaculum thermopropionicum_SI; Petrotoga mobilis SJ95; Polaromonas naphthalenivorans CJ2; Polaromonas sp. JS666; Pseudoalteromonas haloplanktis TAC125; Streptomyces pristinaespiralis ATCC 25486; Streptomyces pristinaespiralis ATCC 25486; Streptococcus thermophilus; Streptomyces viridochromogenes DSM 40736; Streptosporangium roseum DSM 43021; Synechococcus sp. PCC 7335; and Thermosipho africanus TCF52B (Chylinski et al., RNA Biol., 2013; 10 (5): 726-737).

The invention will be further described in the following examples, which do not limit the scope of the invention described in the claims.

### EXAMPLES

### Example 1: Targeted Integration of Insertion Templates in the Albumin Gene in Hepatocytes at a First Target Site

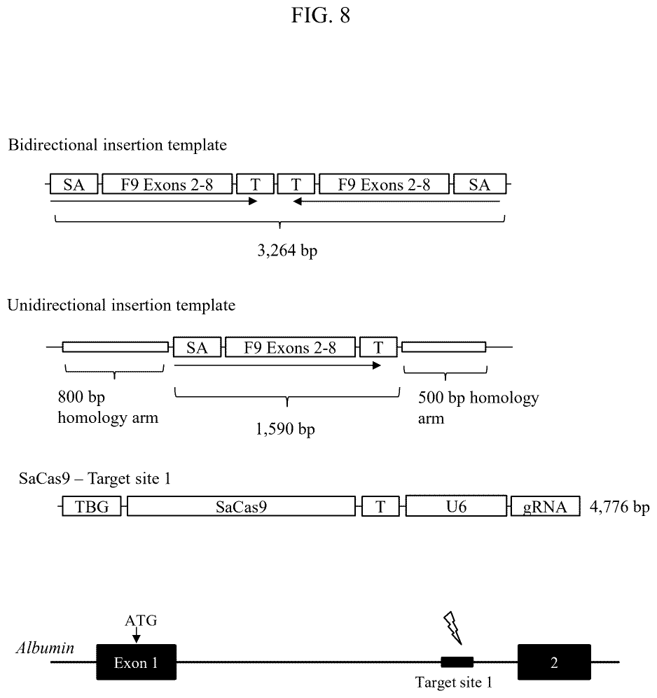

The Mus musculus albumin gene, intron 1, was chosen as the target for insertion. The sequence of intron 1 is shown in SEQ ID NO:1. A first SaCas9 gRNA was designed to target the 21 nucleotide sequence GATCGGGAACTGGCATCTTCA (SEQ ID NO: 2). The complete target site, including the PAM sequence, is GATCGGGAACTGGCATCTTCAGGGAGT (SEQ ID NO: 3). SaCas9-gRNA sequence was cloned within an AAV vector and packaged into AAV8 particles (FIG. 8).

Both uni-directional and bidirectional insertion templates were designed for insertion into intron 1 of the albumin gene. The bidirectional insertion template comprised an [SA]-[CDS]-[T]-[T]-[CDS]-[SA] sequence for insertion via the NHEJ pathway. The uni-directional insertion template comprised [SA]-[CDS]-[T] insert sequence flanked by homology arms (FIG. 8). Both the bidirectional and uni-directional templates were designed to carry exons 2-8 of the Factor 9 (F9) coding sequence. The insertion templates were cloned within AAV vectors and packaged into AAV8 particles.

6-8-week-old male C57BL/6 mice were administered AAV particles by tail-vein injection. Insertion template AAV particles were administered at 7E+11 GC/mouse each, and Cas9 AAV particles were administered at 1.4E+11 GC/mouse. Blood was collected 28 days post injection.

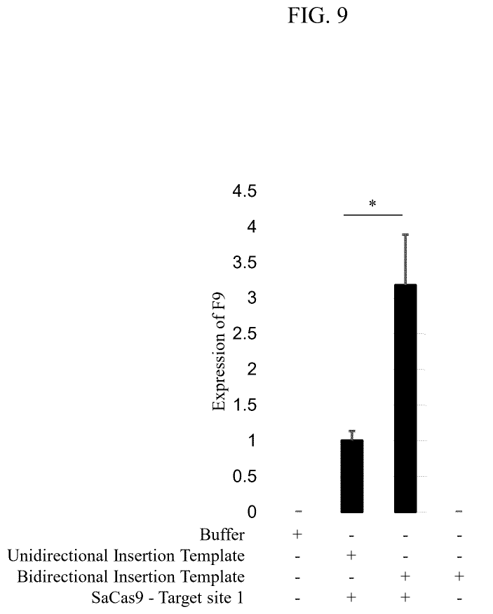

Successful modification of the endogenous gene was predicted to result in expression of exons 2-8 of the F9 coding sequence. F9 protein levels were detected and quantified by ELISA (FIG. 9). These data show the successful targeting of insertion templates into a first gRNA target site within intron 1 of albumin.

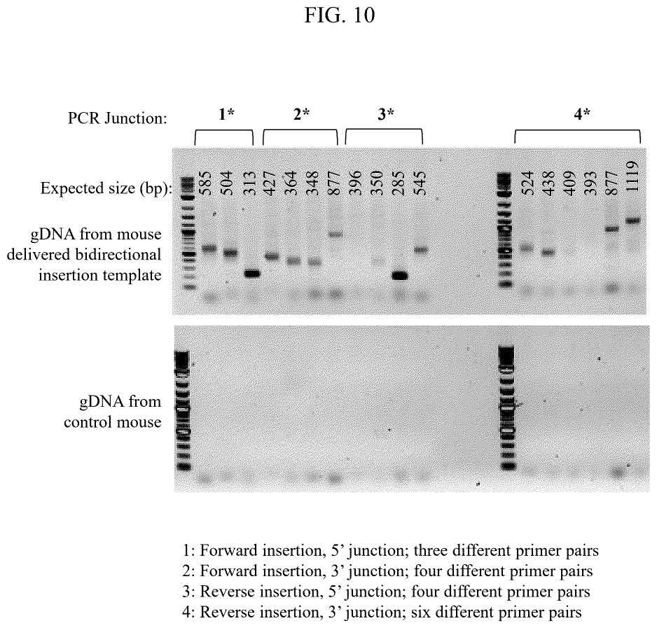

Genomic DNA was extracted from mouse livers 28 days post injection and insertion events were assessed. Junction PCRs were performed on samples administered the bidirectional insertion template. Both forward and reverse insertion events were detected and amplified with PCR (FIG. 10).

### Example 2: Targeted Integration of Insertion Templates in the Albumin Gene in Hepatocytes at a Second Target Site

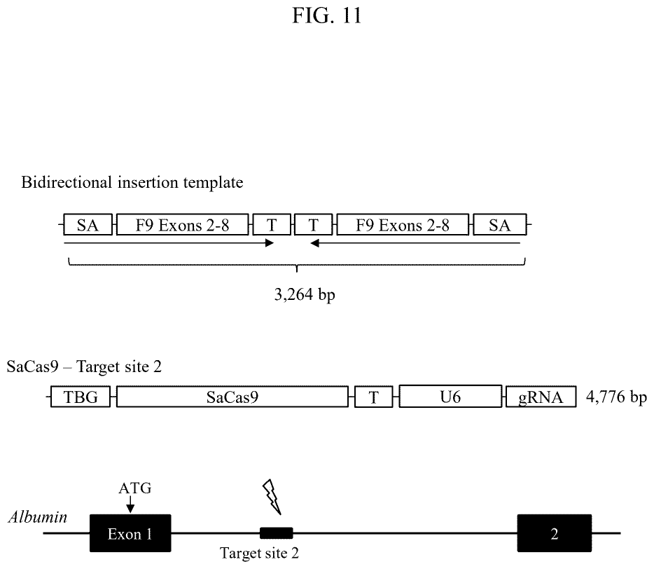

A second SaCas9 gRNA was designed to target the 21 nucleotide sequence ACATGACAGAAACACTAAATC (SEQ ID NO:4). The complete target site, including the PAM sequence, is ACATGACAGAAACACTAAATCTTGAGT (SEQ ID NO:5). SaCas9-gRNA sequence was cloned within an AAV vector and packaged into AAV8 particles (FIG. 11).

To verify successful insertion of the bidirectional template (the same as used in Example 1) into the second target site, 6-8-week-old male C57BL/6 mice were administered AAV particles by tail-vein injection. Insertion template AAV particles were administered at 7E+11 GC/mouse each, and Cas9 AAV particles were administered at 1.4E+11 GC/mouse. Blood was collected 28 days post injection. F9 protein levels were detected and quantified by ELISA, indicating successful targeting of insertion templates into the second gRNA target site within intron 1 of albumin.

### Example 3: Targeted Integration of Insertion Templates within the Albumin Gene in Hepatocytes at a First Target Site Followed by Targeted Integration of Insertion Templates in the Albumin Gene at a Second Target Site

To demonstrate redosing of insertion-based gene editing, 6-8-week-old male C57BL/6 mice are administered SaCas9 AAV particles targeting the first target site, along with the bidirectional insertion template AAV particles. Insertion template AAV particles are administered at 7E+11 GC/mouse each, and Cas9 AAV particles are administered at 1.4E+11 GC/mouse.

Groups of mice are redosed at day 28, day 56, or day 100, following the initial dose. Redosing is achieved by administering SaCas9 targeting the second target site, along with the bidirectional insertion template AAV particles. Insertion template AAV particles are administered at 7E+11 GC/mouse each, and Cas9 AAV particles are administered at 1.4E+11 GC/mouse.

Blood is collected from mice 1) 48 hours prior to redosing, and 2) 28 days post redosing. F9 protein levels are detected and quantified by ELISA. Increased levels of F9 protein levels 28 days post redosing, as compared to F9 protein levels prior to redosing, indicates successful sequential gene insertion events within a population of cells.

### Example 4: Targeted Integration of Insertion Templates within the Albumin Gene in Hepatocytes at a First Target Site Followed by Targeted Integration of Insertion Templates in the Albumin Gene at a Second Target Site

To demonstrate redosing of insertion-based gene editing, mice comprising the human albumin locus are administered SpCas9/gRNA using lipid nanoparticles and a bidirectional template within AAV particles. Two SpCas9/gRNA endonucleases are designed to target a first and second target site within the human albumin gene, intron 1. A first SpCas9/gRNA is designed to a target TAAAGCATAGTGCAATGGATAGG, (SEQ ID NO: 6), which includes the PAM sequence. A second SpCas9/gRNA is designed to target GAGCAACCTCACTCTTGTCTGGG (SEQ ID NO:7), which includes the PAM sequence. The bidirectional template comprises a [SA]-[F9 exons 2-8 CDS]-[T]-[T]-[F9 exons 2-8 CDS]-[SA] sequence for insertion via the NHEJ pathway. The bidirectional template is loaded on an AAV vector and packaged into AAV8 particles.

SpCas9 and the corresponding gRNAs are synthesized as mRNA or RNA by in vitro transcription. SpCas9 mRNA and gRNA are loaded into lipid nanoparticles comprising ((9Z,12Z)-3-((4,4-bis(octyloxy) butanoyl)oxy)-2-((((3-(diethylamino) propoxy) carbonyl)oxy)methyl) propyl octadeca-9,12-dienoate, also called 3-((4,4-bis(octyloxy) butanoyl)oxy)-2-((((3-(diethylamino) propoxy) carbonyl)oxy)methyl) propyl(9Z,12Z)-octadeca-9,12-dienoate), cholesterol, DSPC, and PEG2k-DMG.

Mice are first administered, via tail vein injection, the first SpCas9 targeting the first target site, along with the bidirectional template within AAV particles. Groups of mice are redosed at day 28, day 56, or day 100, following the initial dose. Redosing is achieved by administering SpCas9 targeting the second target site, along with the bidirectional insertion template within AAV particles.

Blood is collected from mice 1) 48 hours prior to redosing, and 2) 28 days post redosing. F9 protein levels are detected and quantified by ELISA. Increased levels of F9 protein levels 28 days post redosing, as compared to F9 protein levels prior to redosing, indicates successful sequential gene insertion events within a population of cells.

### Example 5: Targeted Integration of Insertion Templates within the RHO Gene in Retinal Cells at a First Target Site Followed by Targeted Integration of Insertion Templates in the RHO Gene at a Second Target Site

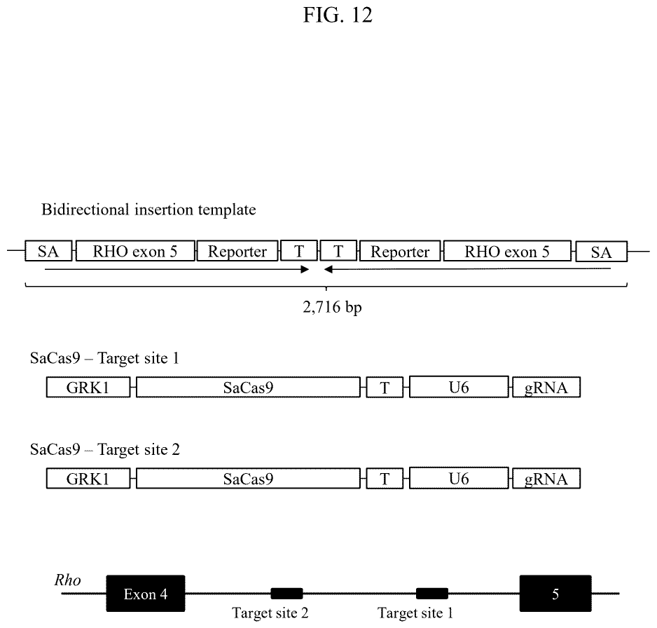

Two SaCas9/gRNA endonucleases targeting a first and second target site within intron 4 of the RHO gene were designed. A first SaCas9 gRNA was designed to target the 21 nucleotide sequence AAGTGGCATTCCATGTGTCCT (SEQ ID NO: 8). A second SaCas9 gRNA was designed to target the 21 nucleotide sequence GTGAGTGATCTCAATTGATTT (SEQ ID NO: 9). The two SaCas9/gRNA sequences were cloned within AAV vectors and packaged into AAV5 particles (FIG. 12). The promoter for expression in retina cells was GRK1.

A bidirectional insertion template was designed for insertion into intron 4 of the RHO gene. The bidirectional insertion template comprised an [SA]-[RHO Exon 5]-[Reporter]-[T]-[T]-[Reporter]-[RHO Exon 5]-[SA] sequence for insertion via the NHEJ pathway (FIG. 12). The insertion templates were cloned within AAV vectors and packaged into AAV5 particles.

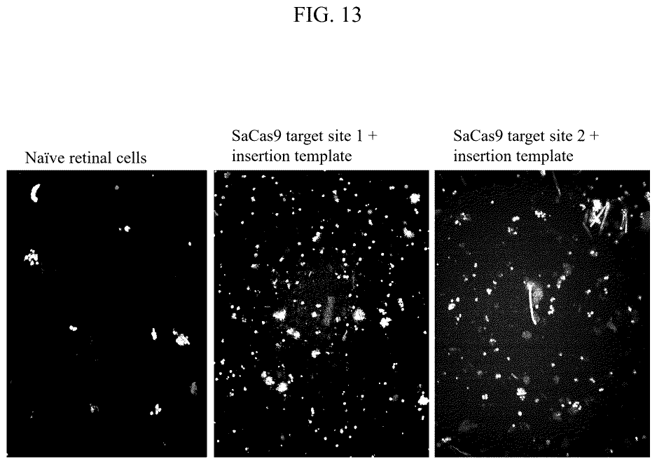

6-8-week-old male Balb/c mice were injected, via subretinal injection, with AAV particles comprising SaCas9 targeting the first target site and AAV particles comprising the bidirectional insertion template. Insertion template AAV particles were administered at 1.5E+9 GC/mouse eye, and Cas9 AAV particles were administered at 1.5E+9 GC/mouse eye. Retinal cells were collected six weeks post injection and assessed for reporter expression via fluorescent microscopy. Imaging revealed reporter gene expression, indicating that the bidirectional template was successfully integrated into target site 1 of the RHO gene (FIG. 13).

6-8-week-old male Balb/c mice were injected, via subretinal injection, with AAV particles comprising SaCas9 targeting the second target site and AAV particles comprising the bidirectional insertion template. Insertion template AAV particles were administered at 1.5E+9 GC/mouse eye, and Cas9 AAV particles were administered at 1.5E+9 GC/mouse eye. Retinal cells were collected six weeks post injection and assessed for reporter expression via fluorescent microscopy. Imaging revealed reporter gene expression, indicating that the bidirectional template was successfully integrated into target site 2 of the RHO gene (FIG. 13).

To demonstrate redosing of insertion-based gene editing, 6-8-week-old male Balb/c mice are injected, via subretinal injection, with AAV particles comprising SaCas9 targeting the first target site and AAV particles comprising the bidirectional insertion template. Insertion template AAV particles are administered at 1.5E+9 GC/mouse eye, and Cas9 AAV particles are administered at 1.5E+9 GC/mouse eye.

Groups of mice are redosed at six weeks or day 100 following the initial dose. Redosing is achieved by via subretinal injection with AAV particles comprising SaCas9 targeting the second target site and AAV particles comprising the bidirectional insertion template. Insertion template AAV particles are administered at 1.5E+9 GC/mouse eye, and Cas9 AAV particles are administered at 1.5E+9 GC/mouse eye. An increase in the number of cells with reporter gene expression after redosing, as compared to the number of cells prior to redosing, indicates successful increase in the frequency of cells with the desired insertion events.

### Example 6: Targeted Integration of Insertion Templates in the Beta-Actin Gene within Central Nervous System Cells at a First Target Site Followed by Targeted Integration of Insertion Templates in the Beta-Actin Gene at a Second Target Site

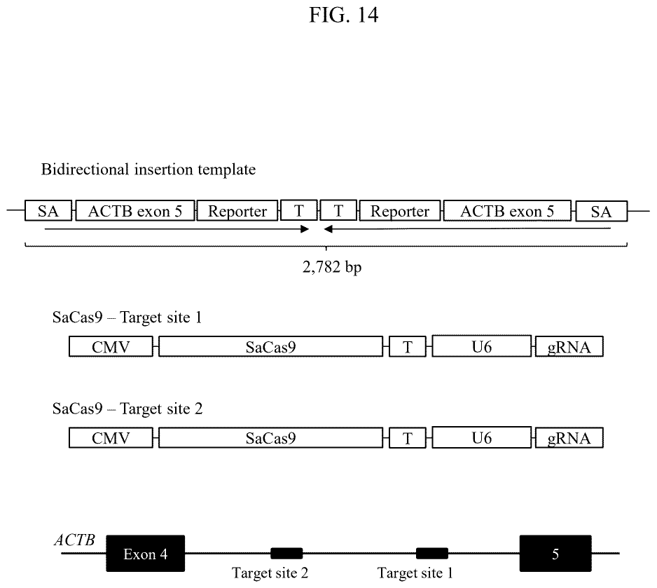

Two SaCas9/gRNA endonucleases targeting a first and second target site within intron 4 of the ACTB gene were designed. A first SaCas9 gRNA was designed to target the 21 nucleotide sequence GAGCAACCTTCTAGGTTTAAG (SEQ ID NO: 10). A second SaCas9 gRNA was designed to target the 21 nucleotide sequence GCTAAGCATCCTTAGCTTGGT (SEQ ID NO: 11). The two SaCas9/gRNA sequences were cloned within AAV vectors and packaged into AAV9 particles (FIG. 14). The promoter for expression in retina cells was CMV.

A bidirectional insertion template was designed for insertion into intron 4 of the ACTB gene. The bidirectional insertion template comprised an [SA]-[ACTB Exon 5]-[Reporter]-[T]-[T]-[Reporter]-[ACTB Exon 5]-[SA] sequence for insertion via the NHEJ pathway (FIG. 14). The insertion templates were cloned within AAV vectors and packaged into AAV9 particles. As a control for transfection, AAV9 particles comprising an AAV vector with a GFP control were also generated.

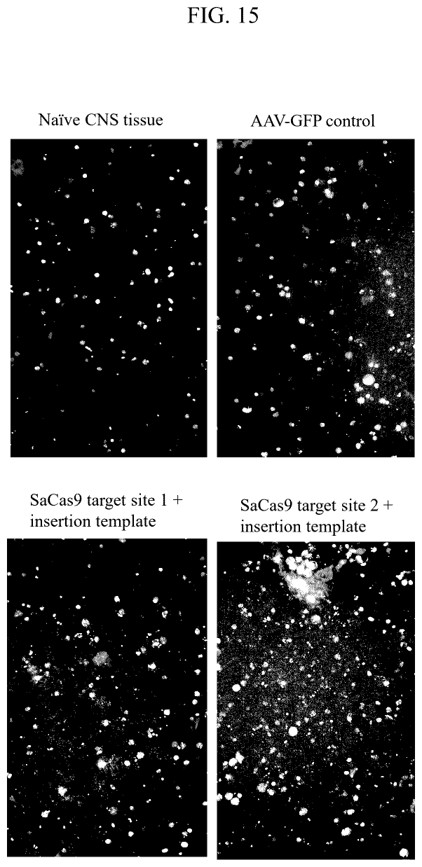

6-8-week-old male C57Bl/6 mice were injected, via intracranial injection, with AAV particles comprising SaCas9 targeting the first target site and AAV particles comprising the bidirectional insertion template. Insertion template AAV particles were administered at a concentration of 1.9E+13 GC, and Cas9 AAV particles were administered at a concentration of 9.2E+12. Approximately 1-2 ul of total volume was administered via intracranial injection. Brain cortex cells were collected 21 days post injection and assessed for reporter expression via fluorescent microscopy. Imaging revealed reporter gene expression, indicating that the bidirectional template was successfully integrated into target site 1 of the ACTB gene (FIG. 15).

6-8-week-old male C57Bl/6 mice were injected, via intracranial injection, with AAV particles comprising SaCas9 targeting the second target site and AAV particles comprising the bidirectional insertion template. Insertion template AAV particles were administered at a concentration of 1.9E+13 GC, and Cas9 AAV particles were administered at a concentration of 9.2E+12. Approximately 1-2 ul of total volume was administered via intracranial injection. Brain cortex cells were collected 21 days post injection and assessed for reporter expression via fluorescent microscopy. Imaging revealed reporter gene expression, indicating that the bidirectional template was successfully integrated into target site 2 of the ACTB gene (FIG. 15).

To demonstrate redosing of insertion-based gene editing, 6-8-week-old male C57Bl/6 mice are injected, via intracranial injection, with AAV particles comprising SaCas9 targeting the first target site and AAV particles comprising the bidirectional insertion template. Insertion template AAV particles are administered at a concentration of 1.9E+13 GC, and Cas9 AAV particles are administered at a concentration of 9.2E+12. Approximately 1-2 ul of total volume is administered via intracranial injection.

Groups of mice are redosed at 21 days or 100 days following the initial dose. Redosing is achieved by via intracranial injection with AAV particles comprising SaCas9 targeting the second target site and AAV particles comprising the bidirectional insertion template. Insertion template AAV particles are administered at a concentration of 1.9E+13 GC, and Cas9 AAV particles are administered at a concentration of 9.2E+12. Approximately 1-2 ul of total volume is administered via intracranial injection. An increase in the number of cells with reporter gene expression after redosing, as compared to the number of cells prior to redosing, indicates successful increase in the frequency of cells with the desired insertion events.

Intron 1 of the mouse albumin gene;
SEQ ID NO: 1
GTAAGAGTTTTATGTTTTTTCATCTCTGCTTGTATTTTTCTAGTAATGG
AAGCCTGGTATTTTAAAATAGTTAAATTTTCCTTTAGTGCTGATTTCTA
GATTATTATTACTGTTGTTGTTGTTATTATTGTCATTATTTGCATCTGA
GAACCCTTAGGTGGTTATATTATTGATATATTTTTGGTATCTTTGATGA
CAATAATGGGGGATTTTGAAAGCTTAGCTTTAAATTTCTTTTAATTAAA
AAAAAATGCTAGGCAGAATGACTCAAATTACGTTGGATACAGTTGAATT
TATTACGGTCTCATAGGGCCTGCCTGCTCGACCATGCTATACTAAAAAT
TAAAAGTGTGTGTTACTAATTTTATAAATGGAGTTTCCATTTATATTTA
CCTTTATTTCTTATTTACCATTGTCTTAGTAGATATTTACAAACATGAC
AGAAACACTAAATCTTGAGTTTGAATGCACAGATATAAACACTTAACGG
GTTTTAAAAATAATAATGTTGGTGAAAAAATATAACTTTGAGTGTAGCA
GAGAGGAACCATTGCCACCTTCAGATTTTCCTGTAACGATCGGGAACTG
GCATCTTCAGGGAGTAGCTTAGGTCAGTGAAGAGAAGAACAAAAAGCAG
CATATTACAGTTAGTTGTCTTCATCAATCTTTAAATATGTTGTGTGGTT
TTTCTCTCCCTGTTTCCACAG
gRNA target site;
SEQ ID NO: 2
GATCGGGAACTGGCATCTTCA
gRNA target site;
SEQ ID NO: 3
GATCGGGAACTGGCATCTTCAGGGAGT
gRNA target site;
SEQ ID NO: 4
ACATGACAGAAACACTAAATC
gRNA target site;
SEQ ID NO: 5
ACATGACAGAAACACTAAATCTTGAGT
gRNA target site;
SEQ ID NO: 6
TAAAGCATAGTGCAATGGATAGG
gRNA target site;
SEQ ID NO: 7
GAGCAACCTCACTCTTGTCTGGG
gRNA target site;
SEQ ID NO: 8
AAGTGGCATTCCATGTGTCCT
gRNA target site;
SEQ ID NO: 9
GTGAGTGATCTCAATTGATTT
gRNA target site;
SEQ ID NO: 10
GAGCAACCTTCTAGGTTTAAG
gRNA target site;
SEQ ID NO: 11
GCTAAGCATCCTTAGCTTGGT

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1) A method of treating a genetic disease in a subject comprising a population of cells comprising a first and second target site in their genome, the method comprising:
a. administering to the subject a first composition comprising an insertion template and a first CRISPR nuclease that targets a first target site;
b. waiting a period of time to allow for the insertion template to integrate into the first target site of a population of cells in the subject, wherein the period of time is between 1 day and 20 years;
c. administering to the subject a second composition comprising the insertion template and a second CRISPR nuclease that targets the second target site, whereby the insertion template is integrated into the genome of the population of cells and/or their progeny at the second target site.

2) The method of claim 1, wherein the second composition is administered 1 day or more after the first composition.

3) The method of claim 1, wherein the second composition is administered 7 days or more after the first composition.

4) The method of claim 1, wherein the second composition is administered 1 month or more after the first composition.

5) The method of claim 1, wherein the second composition is administered 1 year or more after the first composition.

6)-7) (canceled)

8) The method of claim 1, wherein the CRISPR nuclease is CRISPR/Cas9.

9) The method of claim 1, wherein the first target site is within an intron of an endogenous gene within a cell of the subject.

10) The method of claim 1, wherein the second target site is within an intron of an endogenous gene within a cell of the subject.

11) The method of claim 1, wherein the second target site is within the same intron as the first target site.

12) The method of claim 11, wherein the second target site is upstream to the first target site.

13) The method of claim 11, wherein the second target site is downstream to the first target site.

14) The method of claim 11, wherein the second target site is separated from the first target site by at least 1 nucleotide.

15) The method of claim 11, wherein the second target site is separated from the first target site by at least 10 nucleotides.

16) The method of claim 11, wherein the second target site is separated from the first target site by at least 100 nucleotides.

17) The method of claim 11, wherein the second target site is separated from the first target site by at least 1000 nucleotides.

18) The method of claim 1, wherein the first target site is within the first intron of an albumin gene.

19) The method of claim 18, wherein the first target site comprises the sequence TAAAGCATAGTGCAATGGATAGG (SEQ ID NO: 6).

20) The method of claim 1, wherein the first target site is within intron 4 of a rhodopsin (RHO) gene.

21) The method of claim 1, wherein the insertion template comprises a splice acceptor sequence operably linked to a coding sequence.

22) The method of claim 1, wherein the insertion template comprises a coding sequence operably linked to a splice donor sequence.

23)-24) (canceled)

25) The method of claim 21, wherein the coding sequence encodes an amino acid sequence from a functional protein that is defective in said subject with a genetic disease.

26) The method of claim 22, wherein the coding sequence encodes an amino acid sequence from a functional protein that is defective in said subject with a genetic disease.
